(19) United States
(12) Patent Application Publication
(10) Pub. No.: US 2025/0161058 A1
(43) Pub. Date: May 22, 2025

(54) SURGICAL COUPLING DEVICE

(71) Applicant: The Feinstein Institutes for Medical Research, Manhasset, NY (US)

(72) Inventors: Todd Goldstein, Abertson, NY (US); Stanley E. Asnis, Great Neck, NY (US)

(21) Appl. No.: 18/952,629

(22) Filed: Nov. 19, 2024

Related U.S. Application Data
(60) Provisional application No. 63/600,776, filed on Nov. 20, 2023.

(51) Int. Cl. A61F 2/36; A61B 17/00; A61B 17/72; A61F 2/30

(52) U.S. Cl. CPC A61F 2/3672; A61B 17/72; A61B 2017/00486; A61F 2002/30197; A61F 2002/30235; A61F 2002/30332; A61F 2002/30433; A61F 2002/30448; A61F 2002/30738; A61F 2002/3674

(57) ABSTRACT

A device is used to treat periprosthetic fractures, such as a fracture below the stem of a total hip replacement (THR). The device includes a clamp that is fixed to the exposed stem of the THR and a coupling, such as a morse taper, extending distally away from the stem. A post is inserted into the canal of the distal femur below the fracture. The post has a coupling extending proximally away from the distal femur. The couplings on the clamp and post are joined to create a stable connection between the patient's hip and distal femur. Instead of connecting the clamp with the post directly, one or more adapters may be provided to adjust the length and angulation of the treated limb. The device may also connect a THR stem with a Total Knee Arthroplasty (TKA) that includes a stem.

### REFERENCE TO RELATED APPLICATIONS

This application claims priority under 35 U.S.C. § 119 to U.S. Provisional Patent Application No. 63/600,776, filed on Nov. 20, 2023. The disclosure of that application is incorporated herein by reference.

### BACKGROUND

### Field

This disclosure relates to devices and methods for treating periprosthetic fractures. In particular, the disclosure relates to a device for extending the stem of a prosthesis to fix the prosthesis with healthy, unfractured stable bone. For example, such a device may be used to treat a fracture above or below the stem of a hip replacement prosthesis by fixing the stem with the unfractured bone in the distal femur.

Joint replacement procedures such as a total hip replacement (THR) generally include providing an acetabular component to the patient's pelvis and a femoral component connected with the patent's femur. The femoral component includes a ball at the superior end that forms the ball-and-socket joint of the hip and a stem that extends into the femoral canal.

A patient may fracture the femur connected with the THR above or below the level of the stem of the THR. This continues to be an increasing short and more often long-term complication in Reconstructive Orthopedic Surgery. Rapidly increasing numbers of patients are having replacement procedures. With time and the aging of these patients, there are increased falls in patients with significantly decreased bone density and osteoporosis. In many such cases, the prosthetic joint is working very well before the trauma causing the fracture. The prosthesis may still be well fixed in the metaphysis. The THR itself may be well healed with the pseudo-capsule making the hip stable and the abductors, adductor, and rotator muscles functioning well. Nonetheless, the functioning hip prosthesis may need to be removed in order to treat the fracture.

Treatment of the periprosthetic fracture may be complicated by the stem component of the THR. Using a plate on the diathesis distal to the fracture is not ideal since the THR stem blocks the screws needed to fix the plate proximally to the bone around the stem and only allows uni-cortical screws. Thus, bone screws in the region of the femur above the stem may not be able to be installed bi-cortically. Conventionally for these fractures the femoral component must be removed and a new femoral component with a longer stem used. This takes down the well-functioning hip by removing the pseudo-capsule (like a leather band of scar tissue stabilizing the hip-like a book binding stabilizes the pages of a book). In addition, the femoral component is often cemented or well ingrown. Removal of the femoral component from the metaphysis will often destroy the proximal bone and disrupt the peri-articular muscles. Moreover, once the fracture has been treated, a new THR may be required. This procedure is complicated and subjects the patient to significant trauma. It also may be less stable than the original hip. In addition, such a procedure will require the patient to endure recovery from both the periprosthetic fracture and a replacement THR.

Thus, there is a need for a device that would allow a physician to treat periprosthetic fractures and that allows components of a functioning THR, or other joint replacement prosthetic, to remain in place.

### SUMMARY

According to one aspect of the disclosure, a surgical coupling device connects the stem of a THR or other prosthesis with healthy, unfractured bone. According to another aspect, the device includes selectable components that are assembled to provide a selected length and/or a selected angulation between the stem and the unfractured bone. According to a still further aspect, the device includes adjustable segments that allow a surgeon to modify the length and/or angulation between the stem and the unfractured bone.

According to one embodiment, the device includes a clamp that is fixed with to the stem of a prosthesis, such as a THR prosthetic. One end of the clamp is adapted to surround the end of the stem and to be fixed thereon, for example, by closing a clamshell component against the stem to form a mechanically stable connection. An opposite end of the clamp includes a coupling, such as a male or female morse taper coupling. The device further includes a post. One end of the post includes a morse taper coupling adapted to engage with the coupling of the clamp or with couplings of other components, as will be explained below. The opposite end of the post is shaped to fit within the canal of the femur.

A device within the scope of the disclosure may be used to treat a periprosthetic fracture that has occurred above, at, or below the inferior end of the stem of a THR. The surgeon removes a portion of the proximal femur surrounding the stem of the THR to expose the stem and may remove bone below the fracture to allow access and secure fixation to the distal femur below the fracture. The surgeon fixes the clamp to the exposed stem. The surgeon inserts the post into canal of the distal femur and joins the male and female morse tapers on the clamp and post to create an extended THR prosthesis that now extends from the original stem past the site of the fracture to the distal femur.

A device within the scope of the disclosure may also be used to treat a periprosthetic fracture above a total knee arthroplasty (TKA) that includes a proximally extending femoral stem. The surgeon removes a portion of the distal femur surrounding the stem and fixes the clamp to the exposed stem. The surgeon inserts the post into the canal of the proximal femur and joins the morse tapers on the clamp and post. As a result, the TKA is secured with the proximal femur above the site of the fracture. Likewise, a device within the scope of the disclosure may also be used to treat a periprosthetic fracture below a total knee arthroplasty (TKA) that includes a distal extending tibial stem. The surgeon removes a portion of the proximal tibial surrounding the stem and fixes the clamp to the exposed stem. The surgeon inserts the post into the canal of the distal tibia and joins the morse tapers on the clamp and post. As a result, the TKA is secured with the distal tibia. However, it is contemplated, a device within the scope of the disclosure may be adapted to treat humeral fractures and fractures around a long bone which have been previously rodded.

According to one embodiment, the clamp has a clamshell configuration. Clamshell portions are pulled toward one another by bolts extending between them. The stem of the prosthesis is inserted between the clamshells and the bolts are tightened to form a mechanically stable connection with the stem. The clamp may include set screw openings. Set screws are threaded through the set screw openings and driven into contact with the stem to further secure the clamp to the stem.

According to another embodiment, instead of a clamshell structure, clamp includes an opening sized to accept insertion of the stem and a plurality of set screw openings. Set screws are driven into contact with the stem, thereby fixing the clamp to the stem.

According to a further embodiment, one or more adapters are provided that connect with the clamp and post portions to adjust the length of the extended THR and/or the angular orientation of the proximal and distal portions of the femur to achieve an anatomically correct angulation.

The adapters may each have couplings at each end, for example, morse taper couplings, to joint with respective couplings on the clamp and post and with other extension portions. An adapter with a selected length is used to assemble the device so that, after surgery, the length of the patient's femur is anatomically correct. According to some embodiments, the extension may have morse taper couplings that create a male-to-male, a female-to-female, or a male-to-female interface. According to one embodiment, the device is provided to the surgeon as a kit including a selection of adapters of different lengths. The surgeon adjusts the length of the device by choosing a selected adapter from the kit.

According to another embodiment, adapters include angulation adapters that allow the surgeon to adjust the angulation between components to provide a desired angulation of the patient's limb. The angular adapter has couplings, for example, morse taper couplings at its proximal and distal ends to connect with the clamp, post, and/or other adapters. The proximal and distal couplings define respective proximal and distal axes that are set apart at a selected angle. According to one embodiment, the device is provided to the surgeon as a kit including a selection of angulation adapters at different angles. The surgeon adjusts the angulation by choosing a selected adapter from the kit.

According to another embodiment, one adapter, such as a straight adapter, is provided with one or more keyways and an angulation adapter is provided with a key. When the two adapters are joined, the rotational position of the angulation adapter is fixed by engagement of the key in a selected one of the keyways. This allows the surgeon to lock the rotational connection at a selected position to define a selected articulation.

According to a further embodiment, an adjustable adapter is provided. Couplings at the ends of the adapter are threaded into openings on a rotatable central portion. Rotating the central portion causes the couplings to screw into or out from the central portion to adjust the length of the adapter. According to a further embodiment, the adjustable portion includes a locking mechanism, for example, a set screw, to prevent the threaded engagements from rotating and to fix the device at a selected length.

According to one embodiment, the post includes a distal tapered shaft adapted to fit within the canal of the femur. The shaft may include one or more fixation screw holes adapted to receive reinforcement screws. According to one embodiment, during a procedure to treat a periprosthetic fracture below a THR, the surgeon inserts the shaft of the post into the canal of the distal femur. One or more screws are driven through the femur and into respective fixation screw holes to secure the post to distal femur.

According to another embodiment, the post is fixed with the femur using a layer of bone cement applied to the post prior to being inserted into the femur. According to a further embodiment, the post is hollow. One or more channels are provided through the post to allow bone cement to be injected into the post and to flow to the interface between the post and the inner surface of the canal of the femur.

The post, as well as other components of the device, may include surface features, textures, coatings, or layers adapted to promote bone growth and to facilitate bonding of the post or other components of the device with bony tissue such as the femur.

According to a further embodiment, a device according to the disclosure is adapted to treat a periprosthetic fracture of a patient with both a THR and a TKA that includes a femoral stem. According to this embodiment, two clamps are provided. The surgeon prepares the patient by removing bone fragments surrounding the stems of both the THR and TKA. The clamps are secured to both stems. The male and female morse tapers on the clamps may be selected so that the clamp affixed with the THR and the clamp affixed with the knee prosthetic can be joined directly with one another. According to a further embodiment, one or more adapters, such as described above, are provided between the two clamps.

According to a further embodiment, a porous (or mesh) sleeve is provided to facilitate the connection between the clamp and the stem of a prosthesis. The sleeve is positioned around the stem of the prosthesis. The clamp is fitted over the sleeve. According to one embodiment, the clamp is tightened over the sleeve, deforming the sleeve to conform to the shapes of the clamp and stem. According to one embodiment, the porous sleeve comprises open cells that allow liquid to flow through the space between the clamp and the stem. Once the clamp is fixed with the stem, a liquid or semiliquid adhesive, such as bone cement, is forced into the porous sleeve, filling the pores and creating a secure bond between the clamp and the stem.

According to one embodiment, the porous sleeve is formed from a material that can be trimmed by the surgeon to adjust for variations in the dimensions of the stem and the clamp. According to a further embodiment, the porous sleeve is formed from a biocompatible material using 3-d printing techniques. In use, the surgeon trims the sleeve to a selected length and impacts the sleeve onto the stem. The surgeon then fits the clamp over the sleeve and secures the clamp, for example, by tightening bolts extending through flanges on the clamp so that the clamp is secured onto the sleeve. The surgeon then injects an adhesive into the pores of the sleeve. According to a further embodiment, the surgeon inserts set screw through openings on the clamp to engage with the stem and/or the sleeve. By providing a porous sleeve, a device according to this embodiment assures that the adhesive will be well distributed between the clamp and the bone. The adhesive forms a matrix around the sleeve material to create a composite structure. According to one embodiment, the adhesive-impregnated sleeve better distributes the load applied by the clamp and/or set screws to reduce stress concentrations where screws of components of the clamp impact on the stem.

According to a further embodiment, one or more components of the adhesive, such as the activator of a two-component adhesive system, are incorporated in the porous sleeve prior to use. In use, the surgeon impacts the sleeve onto the stem and connects the clamp, as discussed above. The surgeon then injects a second component of the adhesive, for example, a monomer of the two-component system, that reacts with the activator to bond the clamp to the stem. According to a further embodiment, porous material sheets (material similar to that to make the sleeves) is supplied for the surgeon to shape by cutting, trimming, bending, or compressing to a desired shape then be used in places in the reconstruction that aid in fixation. This material may be or not be reinforced with cement.

According to a further embodiment, one or more of the clamp, adapters, and post include reinforcement openings to connect with reinforcing screws and allow the surgeon to connect reinforcing plates and other fixation prosthetics to the device and to adjacent bone. After installing the device according to any of the embodiments above, the surgeon applies a reinforcing plate. According to one embodiment, such a plate is applied between the proximal or distal femur and the device with one or more bone fragments positioned between the device and the plate. This can add rotational stability to the system or capture large butterfly fragments. Bone screws connect the device to the femur and reinforcing screws pass through the reinforcing plate and engage with the reinforcement openings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a surgical coupling device according to an embodiment of the disclosure connected with the stem of a THR;

FIG. 2 is a view of the device of FIG. 1 with some components disassembled;

FIG. 3 shows a clamp of a surgical coupling device according to another embodiment of the disclosure

FIG. 4 shows the clamp of FIG. 3 connected with the stem of a THR;

FIG. 5 shows periprosthetic fractures below a THR according to their Vancouver Classification and illustrates steps for preparing the bone for treatment;

FIG. 6 shows a surgical coupling device according to another embodiment of the disclosure;

FIG. 7 shows a surgical coupling device of FIG. 6 connected with a THR stem and with a distal femur below a periprosthetic fracture according to an embodiment of the disclosure;

FIG. 8 shows a clamp of a surgical coupling device according to another embodiment of the disclosure;

FIG. 9 shows a clamp of a surgical coupling device according to yet another embodiment of the disclosure;

FIG. 10 shows a post of a surgical coupling device according to an embodiment of the disclosure;

FIG. 11 shows an adapter for a surgical coupling device according to an embodiment of the disclosure;

FIG. 12 shows the configuration of four adapters for a surgical coupling device according to an embodiment of the disclosure;

FIG. 13 shows a device according to an embodiment of the disclosure used to treat a periprosthetic fracture below the stem of a THR and above the stem of a TKA with the components partially disassembled;

FIG. 14 shows the device of FIG. 13 with the components assembled;

FIGS. 15A and 15B show a device for treating a periprosthetic fracture including an adapter that adjusts the angulation of the limb according to embodiments of the disclosure with components partially disassembled in FIG. 15A and assembled in FIG. 15B;

FIG. 16 shows the configuration of four adapters for a surgical coupling device according to another embodiment of the disclosure;

FIG. 17 shows the connection of two adapters according to embodiments of the disclosure that allow adjustment of angulation of a limb being treated for a periprosthetic fracture;

FIG. 18 shows the configuration of two adapters for a surgical coupling device that allow adjustment of length of the device according to embodiments of the disclosure;

FIG. 19 shows the connection between adapters including a crown to facilitate assembly and disassembly of the surgical coupling device according to an embodiment of the disclosure;

FIG. 20 shows a morse taper disassembly tool used to disassemble the adapters of FIG. 19;

FIG. 21 shows a tool for assembling and disassembling morse taper couplings according to an embodiment of the disclosure;

FIG. 22 shows a surgical coupling device for treating a periprosthetic fracture below the stem of a THR and above the stem of a TKA;

FIG. 23 shows a sleeve for facilitating the connection between the clamp of a surgical coupling device and the stem of a prosthetic device according to an embodiment of the disclosure;

FIG. 24 shows the sleeve of FIG. 23 used to facilitate connection of a surgical coupling device with the stem of a THR according to an embodiment of the disclosure;

FIG. 25 shows a 3D printed matrix used to facilitate connection of a surgical coupling device according to an embodiment of the disclosure with the stem of a THR;

FIG. 26 shows another 3D printed matrix used to facilitate connection of a surgical coupling device according to an embodiment of the disclosure; and

FIG. 27 shows an adapter with holes that allow an external plate to be attached to the surgical coupling device and distal femur according to an embodiment of the disclosure.

### DETAILED DESCRIPTION

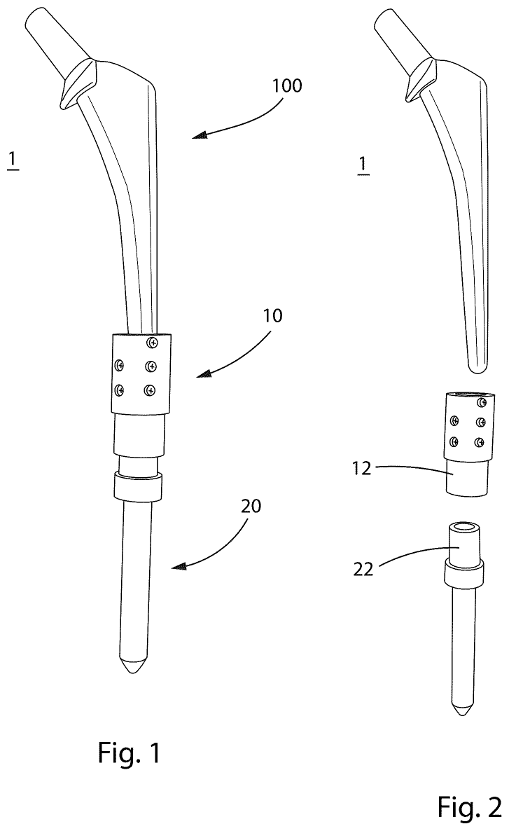

Throughout this disclosure, the surgical coupling device is associated with a prosthetic device of a THR or TKA. However, this is merely for illustrative purposes and this disclosure includes prosthetic devices adapted to treat humeral fractures and fractures around a long bone which have been previously rodded. FIG. 1 shows device 1 according to an embodiment of the disclosure used to treat periprosthetic fractures. FIG. 2 shows device 1 with some components disassembled. According to one embodiment, device 1 is formed by a clamp portion 10 adapted to couple with the stem of a prosthetic device 100, such as the femoral component of a THR or TKA, and that can be fixed with a post portion 20 adapted to be inserted into the unfractured portion of the femur. Couplings 12 and 22 are provided at ends of the clamp portion 10 and post portion 20, respectively, that allow a surgeon to join them once the clamp and post are installed. According to one embodiment, couplings 12, 22 are matched male and female morse taper couplings. In the embodiment shown in FIG. 2, a female coupling 12 is provided on clamp 10 and a male coupling 22 is provided on post 20. The disclosure also encompasses providing the female coupling on the post and the male coupling on the clamp.

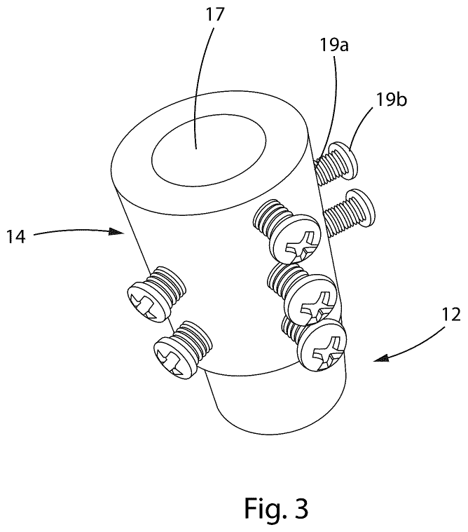

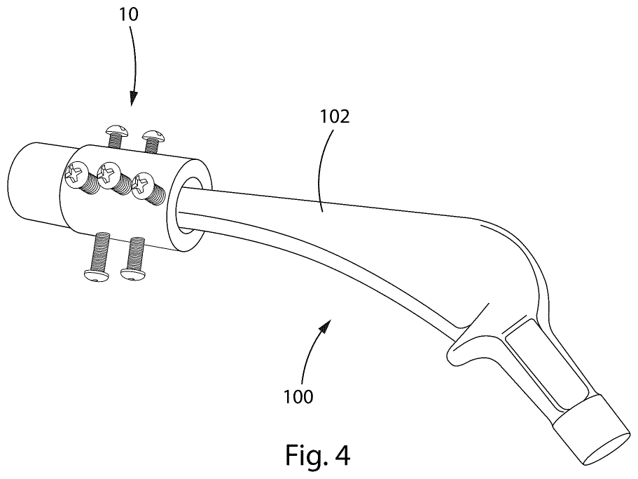

FIG. 3 shows a clamp 10 according to an embodiment of the disclosure. Cavity 17 is formed in clamping mechanism 14 of clamp 10. A plurality of threaded holes 19a are provided through the wall of clamping mechanism 14. A corresponding plurality of bolts are threaded into the holes. FIG. 4 shows clamp 10 connected with stem 102 of prosthesis 100, such as a THR. Cavity 17 is sized and shaped to allow insertion of stem 102 into the cavity. Once the stem is inserted, the surgeon rotates bolts 19b to drive them against the surface of stem 102 to form a mechanically stable connection with prosthesis 100. Cavity 17 may be sized and shaped to allow an angulation of the stem with respect to the device. By tightening selected ones of the bolts 19b, the surgeon can adjust the shape of the stem angulation of the device with respect to the stem and to the patient's anatomy. Cavity 17 is shown with a circular cross section but could be any shape suitable to accept insertion of stem 102. According to one embodiment, cavity 17 is formed in a shape adapted to connect with a selected manufacturer's THR or TKA product.

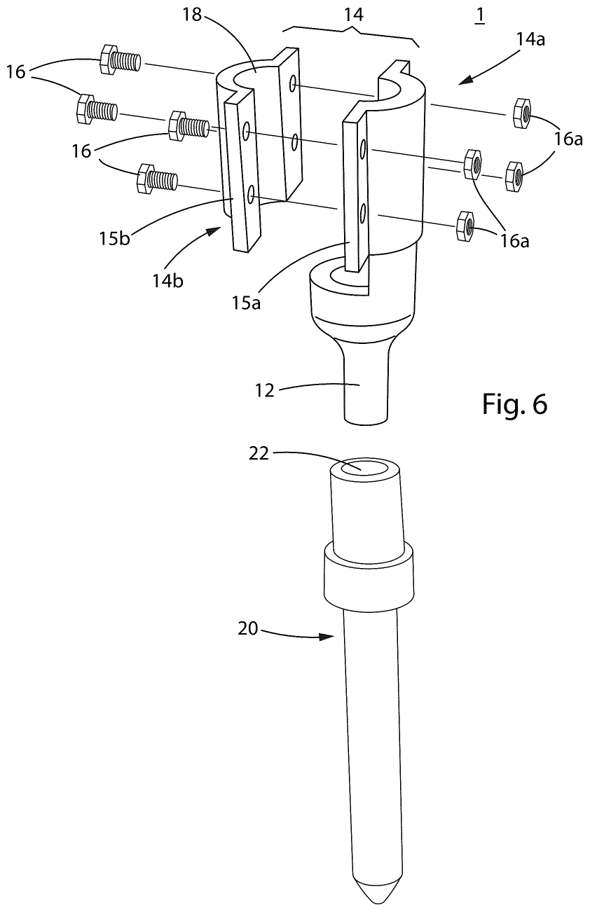

FIG. 6 shows device 1 including clamp 10 according to a further embodiment of the disclosure. Clamping mechanism 14 is formed by a first clamshell 14a connected with coupling 12, and a second clamshell 14b. Clamshells 14a and 14b each have respective flanges 15a, 15b that include one or more bolt holes. Clamshells 14a and 14b are joined with one another by one or more fasteners 16. According to one embodiment, fasteners 16 are bolts that engage into nuts. According to another embodiment,-bolt holes on one or the other flange 15a, 15b are threaded to engage with bolts extending through the opposite flange. When clamshells 14a, 14b are connected, they define a cavity 17 adapted to receive the end of the stem of a prosthetic device, such as on the femoral component of a THR or TKA.

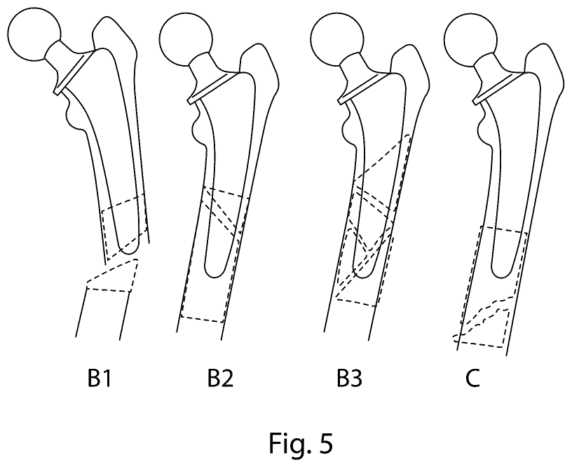

In many periprosthetic fractures, the femur near a THR is fractured but fixation is still good between the proximal metal stem of the femoral component and the bone of the proximal femur and trochanters. FIG. 5 shows periprosthetic fractures associated with a THR as identified by the Vancouver Classification. Depending on the site of the fracture, portions of bone (shown in dashed lines) are removed to expose a sufficient portion of the stem to securely engage with clamp 10. Portions of bone may also be removed below the fracture to allow the surgeon to access the distal femur to install post 9.

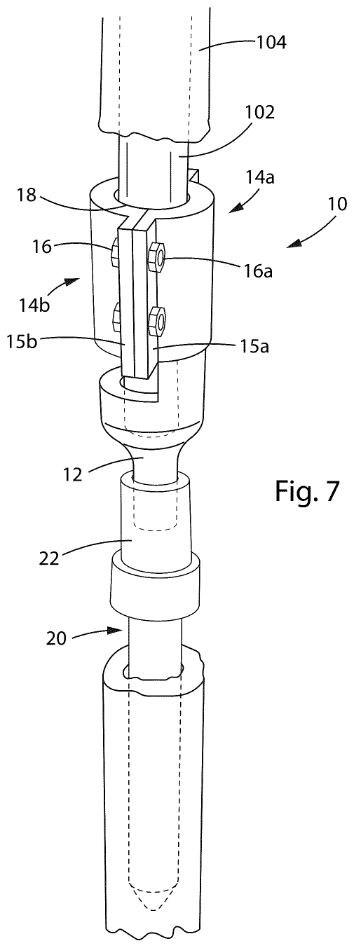

FIG. 7 shows clamp 10 engaged with the distal end of stem 102 of a THR following a periprosthetic fracture. Stem 102 extends through the canal of the proximal femur 104. To the extent necessary, bone has been removed from the femur to expose a portion of stem 102. The distal end of stem 102 is inserted into cavity 17 and clamshells 14a, 14b of clamp 10 are pulled together to apply force onto stem 102 to fix clamp 10 with the stem. Post 20 is inserted into the canal of the distal femur, below the fracture. Post 20 is fixed within the distal femur as will be explained below. Post 20 is joined with clamp 10 by couplings 12, 22 to secure the proximal and distal femurs, thus stabilizing the fracture.

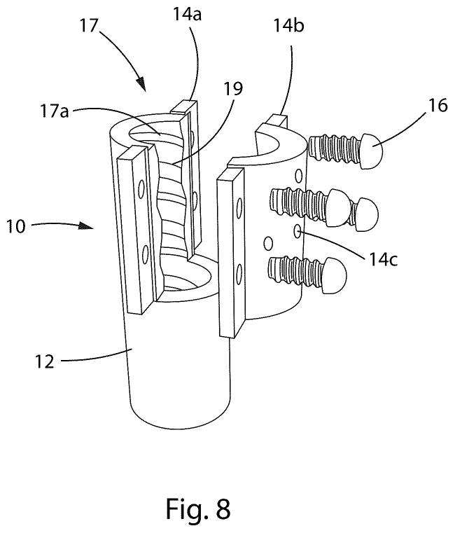

FIG. 8 shows clamp 10 according to a further embodiment of the disclosure. The inner surface 17a of cavity 17 includes features such as ridges adapted to facilitate creating a strong and mechanically stable connection between device 1 and the stem. In addition, the inner surface 17a of cavity 17 may include features 19, such as grooves to facilitate the flow of adhesives, such as bone cement between the stem and clamshells 14a, 14b.

According to one embodiment, one or more ports 14c are provided on one or both of the clamshells 14a, 14b. Ports 14c allow a surgeon to further facilitate the fixing of clamp 10 with the stem. Ports 14c may be used to inject bone cement or other adhesive material into the space between clamshells 14a, 14b and stem 102. In another example, ports 14c are adapted for the use of set screws or other mechanical fasteners. It is contemplated within this disclosure that ports 14c may be used for the injection of bone cement or the like and mechanical fasteners to facilitate the fixing of clamp 10 with the stem.

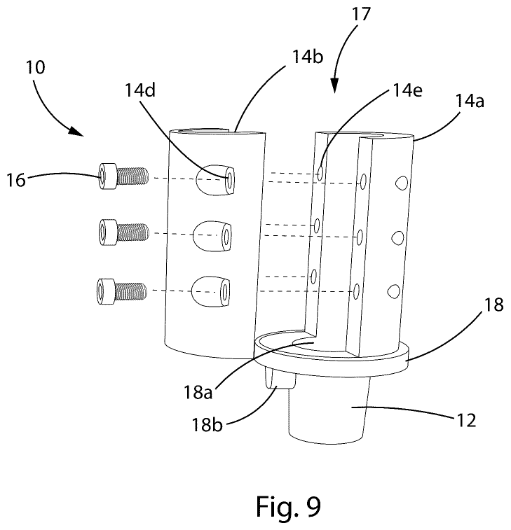

FIG. 9 shows a clamp 10 according to a further embodiment of the disclosure. In this embodiment, no flanges are provided. Instead, clamshell 14b is provided with through holes 14d and clamshell 14a is provided with corresponding threaded holes 14e. One or more bolts 16 are inserted through hole 14d and engage with the threading of holes 14e. Holes 14d may be depressed into the surface of clamshell 14b so that the heads of bolts 16 do not protrude from the surface of the device.

Clamp 10 includes cavity 17 disposed between clamshells 14a, 14b and coupling 12. Crown 18 may define a central passage 18a that extends from cavity 17 through coupling 12. Such a passage allows adhesive to be injected into the space between the stem 102 and clamshells 14a, 14b by way of the end of coupling 12. Crown 18 may also include a key 18b extending from the crown along at least a portion of coupling 12. As will be explained below, key 18b allows clamp 10 to be set at a fixed orientation with respect to other component according to some embodiments of the disclosure.

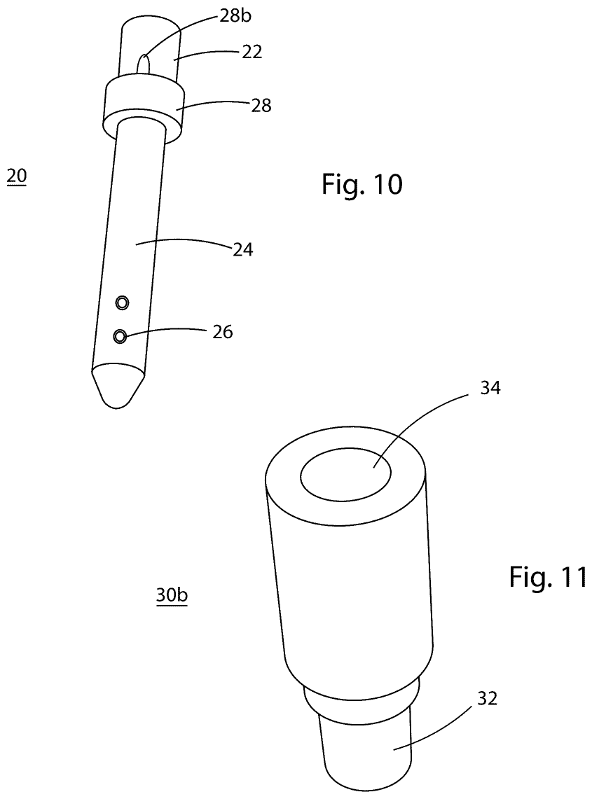

FIG. 10 shows post 20. Coupling 22 connects post 20 with clamp 10 or other components. Crown 28 extends radially outward of coupling 22. Shaft 24 extends away from the crown. Shaft 24 is sized and shaped to be inserted into the canal of a long bone, such as a femur. As discussed above, coupling 22 is adapted to form a mechanically stable connection with clamp 10, or with other components, as will be discussed below. Shaft 24 may have surface features to promote the ingrowth of bone such as coatings, textures, and bone growth promoting materials. It may also be a shape and surface best for being cemented in the femoral shaft.

According to one embodiment, shaft 24 includes one or more threaded reinforcement receiving openings 26. Openings 26 allow a surgeon to install a reinforcing bolt or cross screw through the femur to engage with threads on the openings. Such a bolt may be used to secure post 20 with the distal femur by inserting the bolt through the femur, which may engage the patient's bone uni-cortically or bi-cortically.

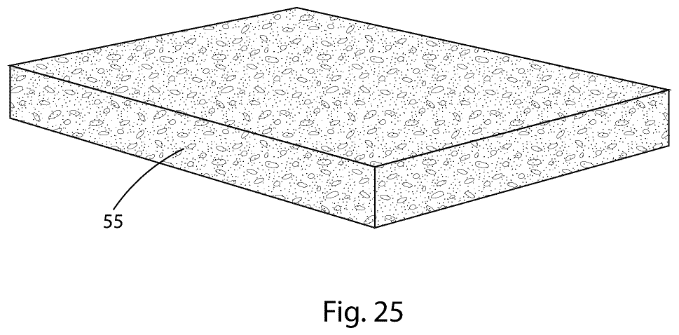

According to another embodiment reinforcement openings 26 may be used to connect plates, rods, or other bone fixation structures. As shown in FIG. 25, plate 300 may be connected with a selected adapter, for example, adapter 30a, which will be discussed in more detail below, (or another component of device 1 equipped with threaded reinforcement receiving openings 26) by screws or bolts 304 passing through plate 300 and threaded with openings 26. Bone screws 306 are provided to connect plate 300 with unfractured bone distal of post 20 (in the case of a THR periprosthetic repair). Because there is no prosthesis in the canal of the distal femur below post 20, bone screws 306 can be inserted bi-cortically.

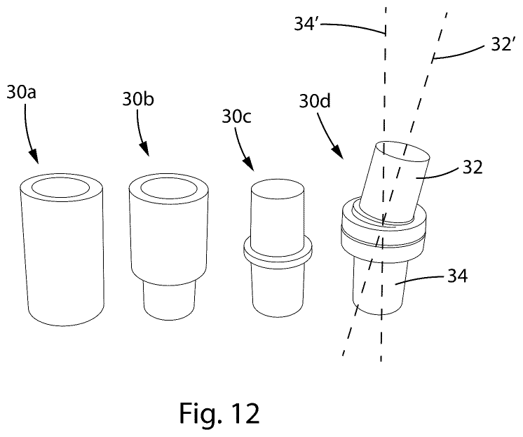

FIG. 11 shows an adapter 30b according to an embodiment of the disclosure. Adapter 30b has couplings 32 and 34 at either end that are adapted to connect with couplings 12 and 22 on the clamp 10 and post 20, respectively, as well as to other components, as will be discussed below. FIGS. 12 shows adapters 30a, 30c, and 30d according to further embodiments of the disclosure. Adapter 30a includes a female morse taper coupling at each end. Adapter 30b has a male coupling on one end and a female coupling on the other. Adapter 30c has male couplings at both ends. Adapter 30d also has male couplings at both set at an angle to adjust angulation, as will be discussed below.

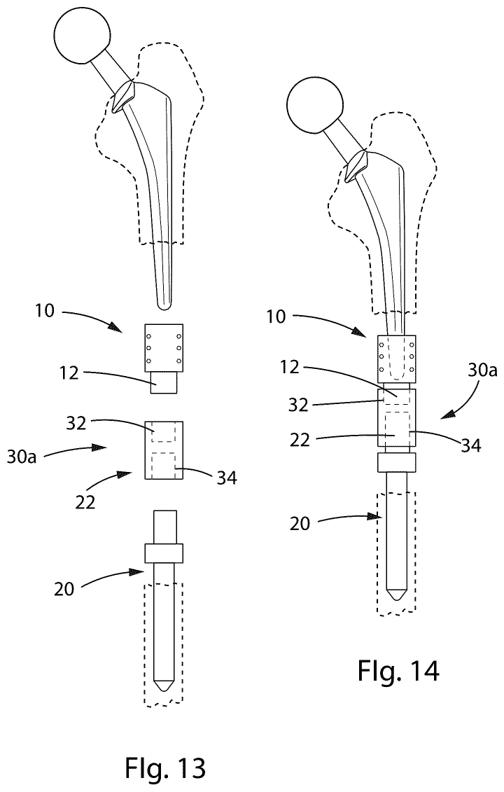

FIGS. 13 and 14 show device 1 according to another embodiment of the disclosure. Adapter 30a is connected with clamp 10 by engagement of couplings 12 and 32 and with post 20 by engagement of couplings 34 and 22. The length of the adapter may be selected to provide sufficient length for the assembled device to provide the correct length of the femur postoperatively. In this embodiment, couplings on the clamp and post 12, 22 are male couplings. Other arrangements are within the scope of the disclosure. For example, adapter 30b, which has a male coupling at one end and a female coupling at the other, would be used to connect a clamp 10 and post 20 where one included a male coupling and the other a female coupling. Adapter 30c, which has a male coupling at both ends, would be used to connect a clamp 10 and post 20 that each have a female coupling. According to another embodiment, device 1 may include multiple adapters 30. For example, two or more adapters 30 could be provided between the clamp and post to create a device with the desired length.

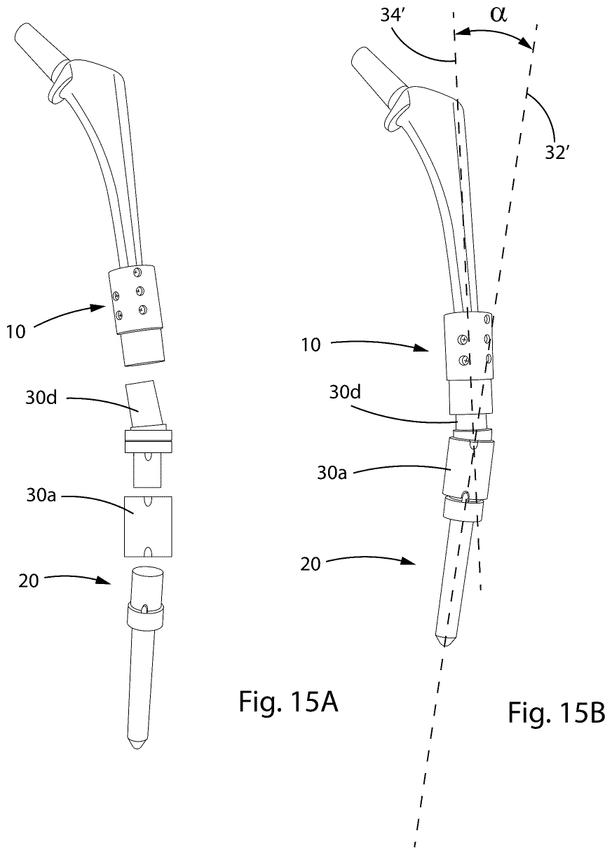

FIGS. 15A and 15B show device 1 according to a further embodiment of the disclosure. FIG. 15A shows device 1 prior to assembly of components. Clamp 10 is engaged with stem 102 of a prosthetic device, which would be in place in the patient's proximal femur. In use, post 20 would be inserted into the canal of a long bone such as a distal femur. Adapters 30a and 30d join the coupling 12 of clamp 10 with coupling 22 of post 20. As shown in FIG. 12, adapter 30d has couplings 32 and 34 at either end. Each such coupling defines a respective coupling axis 32′, 34′, with the coupling axes separated by angle a.

FIG. 15B shows device 1 assembled. In use, a surgeon is provided with a kit including a number of adapters 30d, each with a different angle a. By selecting an adapter 30d, the surgeon can adapt the device to create the desired angulation. In addition, according to one embodiment, each of the components 10, 20, 30 can rotate with respect to one another about their respective couplings. This allows the surgeon to adjust rotation between the proximal and distal femurs before fixing the couplings together.

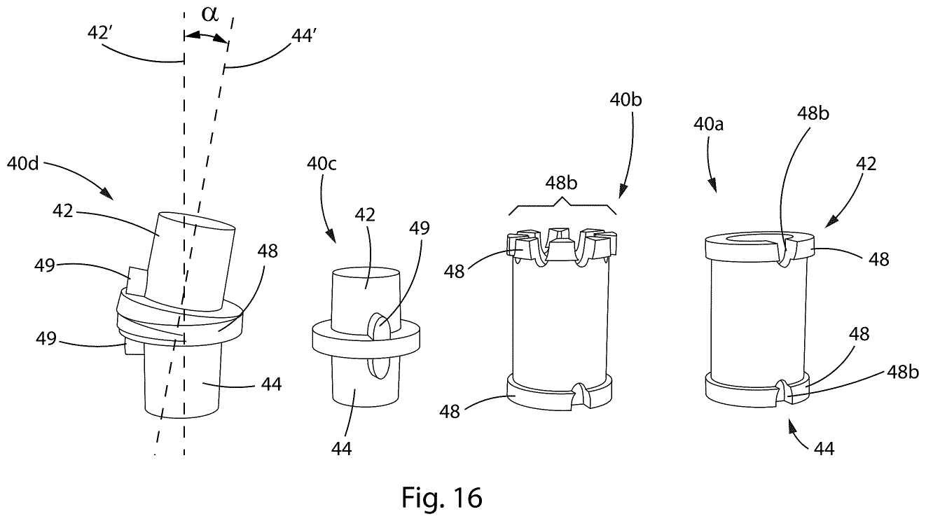

FIG. 16 shows adapters 40a, 40b, 40c, and 40d according to another embodiment of the disclosure. Adapter 40a is similar to adapter 30a discussed above. Female couplings 42, 44 are provided at either end of the adapter. Adapters 40a and 40b includes a keyway 48b shaped to receive key 18b such as key 18b on clamp 10 as shown in FIG. 9 and key 28b of post 20, as shown in FIG. 10. Corresponding keys and keyways may be provided on the posts, adapters, and clamps and other components of the embodiments of the disclosure. Engagement of keys 18b, 28b, with keyways 48b fixes the components rotationally with one another.

As shown in FIG. 16, adapter 40b has female couplings at each end and includes a plurality of keyways 48b on at least one end. Multiple keyways 48b allow other components that include keys to engage with adapter 40b at a selection of fixed rotational positions.

Adapter 40c has male couplings 42, 44 at opposite ends and includes keys 49 that engage with keyways 48, for example, on adapter 40a or 40b to rotationally fix adjacent adapters.

Adapter 40d is used to provide angulation similar to adapter 30d. Adapter 40d has male couplings 42, 44 at each end. As with adapter 30d, each coupling defines a respective coupling axis 42′, 44′ that are at an angle a with one another. Adapter 40d includes keys 49 adjacent to each coupling. Keys are shaped to engage with keyways 48b, for example, on adapter 40a or 40b.

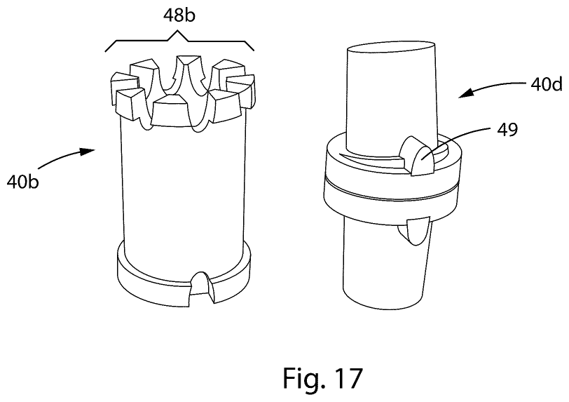

FIG. 17 shows engagement between adapters 40b and 40d. By selecting a coupling 40d with desired angle a and engaging key 49 with a selected one of the plurality of keyways 48b at one end of adapter 40b, the surgeon can select both angulation and orientation of the limb following surgery. Engagement of key 49 with a selected keyway 48b fixes rotation between the components of device 1 and allows the surgeon to choose selected angulation and rotation.

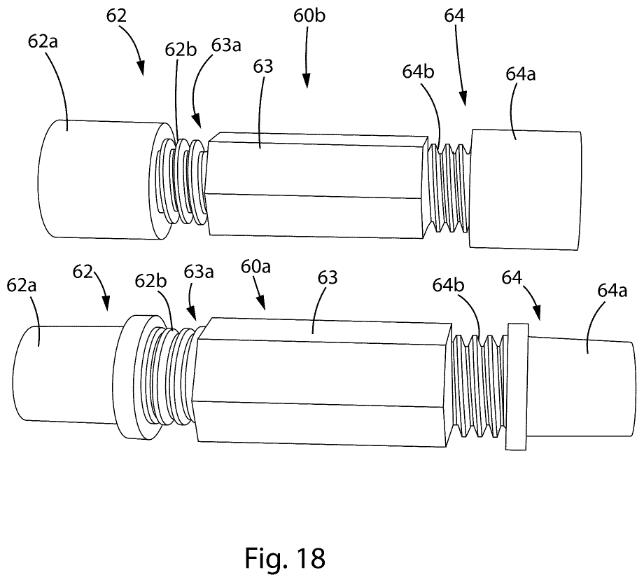

FIG. 18 shows adapters 60a and 60b according to a further embodiment of the disclosure. Adapter 60a includes a tool engagement feature 63 and couplings 62, 64 at either end that are adapted to connect with couplings 12 and 22 on the clamp 10 and post 20, respectively, as well as to other components. Tool engagement feature 63 has a threaded cavity 63a therethrough. In the embodiment shown, the exterior of tool engagement feature 63 is hex shaped and configured to engage a tool, for example, a wrench, for rotating the tool engagement feature 63 in one direction and/or the other direction relative to one or both couplings 62, 64. Other tool interfaces and tools could be used as would be apparent to one of ordinary skill in the art.

In the embodiment of adapter 63a coupling portions 62a, 64a are male taper couplings. Couplings 62, 64 include coupling portions 62a, 64a and threaded portions 62b, 64b. Threaded portions 62b, 64b are configured to engage the threaded cavity 63a. As a surgeon rotates the tool engagement feature 63 in a first direction, the coupling portions 62a and 64a are drawn toward each other, thereby shortening adapter 60a. Likewise, as the surgeon rotates the tool engagement feature 63 in a second direction, the coupling portions 62a and 64a move away from each other, thereby lengthening adapter 60a.

Adapter 60b is substantially similar to adapter 60a, except adapter 60b has female morse taper couplings. It is contemplated in this disclosure that any combination of male and female morse taper couplings may be used. It is also contemplated, that adapters 60a, 60b may include keyways and/or keys as described above referring to adapters 40a, 40b, 40c, and 40d.

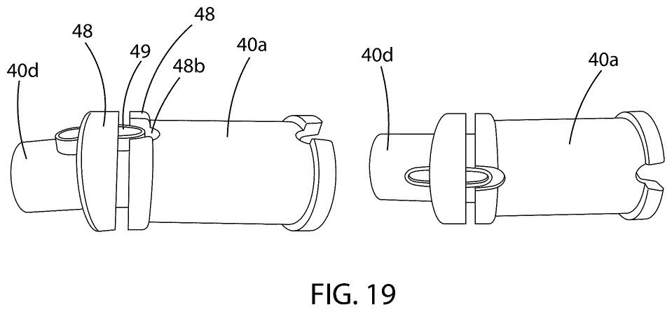

As shown in FIG. 16, adapters 40a, 40b, 40c, and 40d each include a crown 48 disposed adjacent to couplings 42, 44. Clamp 10 and post 20 may likewise include crowns 18, 28 adjacent to their respective couplings 12, 22. FIG. 19 shows engagement of two components, here adapters 40a and 40d, with one another. Crowns 48 of the respective adapters provide a place for a clamp holder or other assembly tool to hold and manipulate the pieces during assembly. Crowns 48 also provides a surface against which compression can be applied to securely connect couplings 42, 44. According to one embodiment, when components, such as adapter 40a and 40d are connected, crowns 48 remain separated from one another by a gap to create space for jaws of a disassembly tool to engage and separate the components.

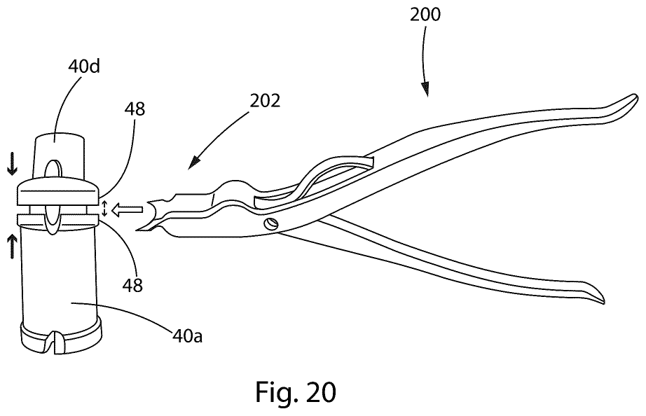

FIG. 20 shows disassembly tool 200 positioned to engage crowns 48 of adapters 40a and 40d. Jaws 202 of tool 200 are inserted between crowns 48. The tool is operated to so that jaws 202 push the adapters 40a, 40d away from one another.

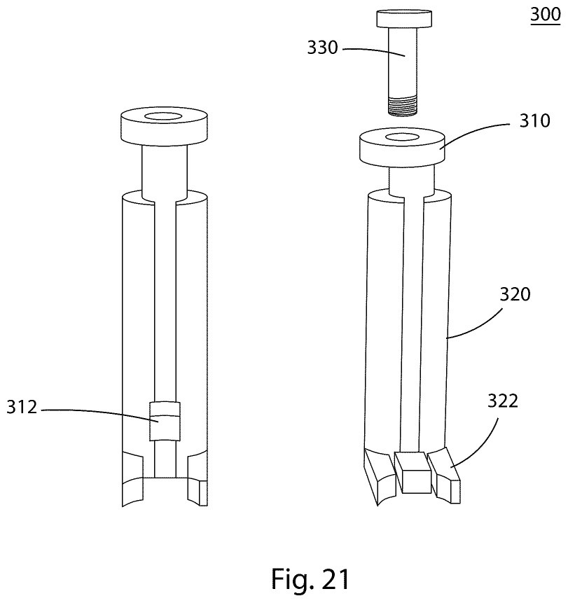

FIG. 21 shows a device for assembling and disassembling components of device 1. Handle component 320 includes lower jaws 322 at a distal end. Slider 310 is received in a cavity within handle portion 320 and can slide freely along the axis of handle portion 320. Slider 310 includes lower jaw 312 that extends outward from handle component 320. To close a morse taper connection, for example, between clamp 10 and post 20, upper jaw 312 is placed above crown 18 of clamp 10 and lower jaws 322 are placed below crown 28 of post 20. Impact is applied to slider 310 by the surgeon to drive couplings 12, 22 into mutual engagement.

To disassemble couplings, impactor 330 is inserted into a cavity within slider 310 and locked with the slider so that upward force on impactor 330 is communicated to slider 310. Lower jaws 322 are placed above crown 28 of post 20 and upper jaw 312 is placed below crown 18 of clamp 10 so that the upper and lower jaws are placed in the gap between the crowns. The surgeon applies impacts to the impactor 330 upward in the orientation of FIG. 21, causing the upper and lower jaws 312, 322 to separate and pull the crowns apart.

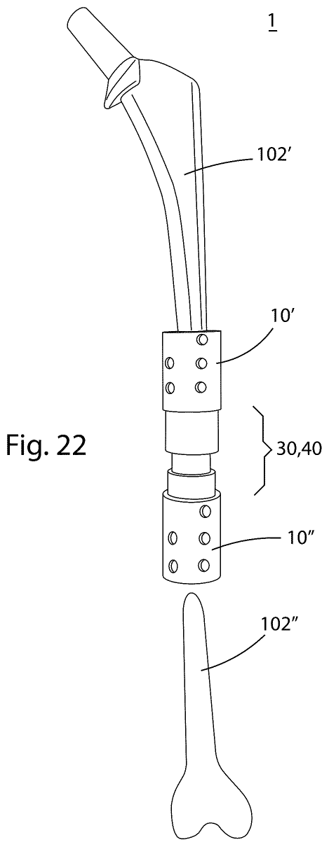

In cases where a patient has had a THR and a TKA and where the TKA includes a femoral stem component 102″, device 1 may be configured with clamps 10 at both ends. FIG. 22 shows device 1 according to an embodiment of the disclosure. Proximal clamp 10′ is connected with the distal end of THR stem 102′. Distal clamp 10″ is connected with the proximal end of stem 102″ of the TKA prosthesis. One or more adapters 30, 40 are provided to connect proximal and distal clamps 10′ 10″ with one another and to adjust the length, rotation, and angulation provided by device 1.

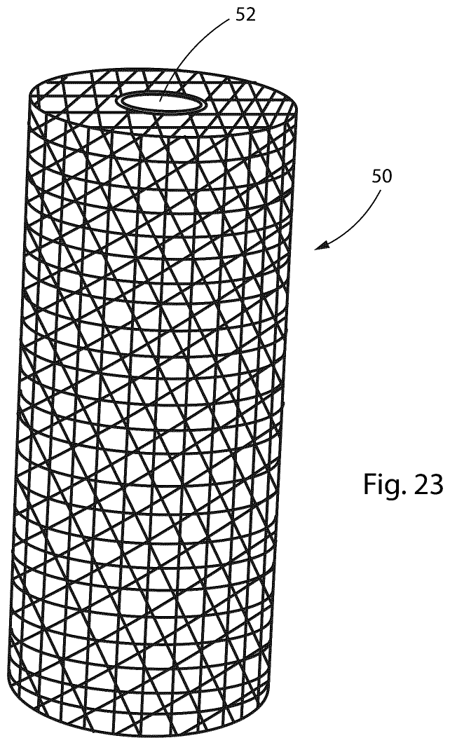

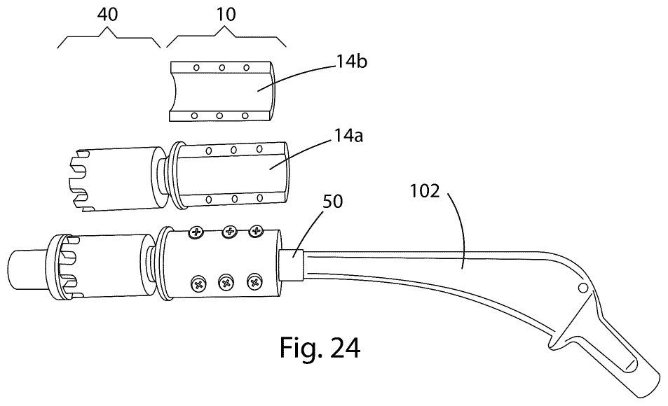

FIG. 23 shows a sleeve 50 according to an embodiment of the disclosure. Central opening 52 is provided at least partially through the center of sleeve 50. FIG. 24 shows sleeve 50 provided between stem 102 of a prosthesis and clamp 10. Sleeve 50 may be formed from a compressible material that readily conforms to the shape of stem 102 to provide a tight fit. According to one embodiment, sleeve 50 can be trimmed or otherwise shaped by the surgeon prior to use.

As shown in FIG. 24, sleeve 50 is impacted over the end of stem 102 prior to connecting clamp 10 with the stem. Stem 102 and sleeve 50 are inserted into cavity 17 and bolts 19b are tightened to compress sleeve 50 against stem 102 to provide a secure engagement with stem 102. According to another embodiment, clamp 10, as shown in FIGS. 6-9, is connected with stem 102 by positioning sleeve 50 between clamshells 14a and 14b and drawing the clamshells together by tightening bolts 16 so that the clamshells compress sleeve 50 to create a secure connection with stem 102.

According to one embodiment, sleeve 50 is porous to allow adhesive, such as bone cement, to be injected into the space between clamp 10 and stem 102. The cement infiltrates the sleeve and solidifies to create a strong composite structure with the adhesive forming a matrix around the porous sleeve material. According to one embodiment, sleeve 50 is formed from a porous metallic mesh, such as tantalum fiberbone. According to another embodiment, sleeve 50 is formed from a polymer, such as polypropylene. According to one embodiment, sleeve 50 is coated with a monomer or the first component of a two-component adhesive system prior to use. Once the surgeon is satisfied that the device is properly installed, the surgeon injects a polymerizing agent or the second component of the adhesive system into the sleeve material to bond the clamp, sleeve, and stem together.

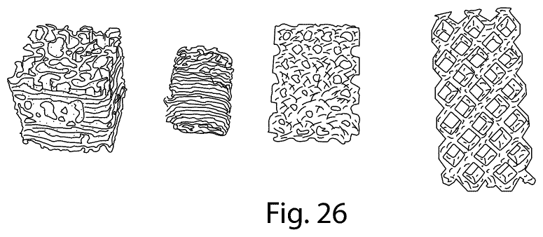

FIGS. 25-26 show a 3D printed matrix 55 according to an embodiment of this disclosure. 3D printed matrix 55 is substantially similar to sleeve 50, as described above, except that 3D printed matrix 55 is configured to be fashioned into any shape as desired by the surgeon. 3D printed matrix 55 may be in the form of a sheet, as shown in FIG. 25, or in a block, as shown in FIG. 26. However, it is contemplated in this disclosure, 3D printed matrix 55 may be of any suitable shape that allows the surgeon to fashion the 3D printed matrix into the desired configuration. 3D printed matrix 55 may be malleable such that a surgeon is able to adjust the shape and form of the 3D printed matrix to achieve optimal fit, alignment, and stability. Non-limiting examples of how the surgeon may shape the 3D printed matrix to the desired configuration include, but is not limited to, cutting, compressing, bending, drilling, and burring. Once the surgeon has the custom shape, for example, to fit between clamp 10 and stem 102, the matrix is embedded in bone cement making a solid shape. The cement can be either with no antibiotics or high dose antibiotics when infection is present. In cases where infection is present or imminent, the matrix can even be PLA so as it dissolves more surface for increased antibiotic release.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

We claim:

1. A device for treating a periprosthetic fracture comprising:
a clamp having a clamping portion to selectively connect the clamp with a stem of a prosthetic device at a first end and a clamp coupling at a second end; and
a post having a bone engagement portion that is sized and shaped to be inserted into a canal of a long bone and a post coupling connected with the bone engagement portion, wherein the clamp coupling and the post coupling are connected.

2. The device of claim 1, wherein the stem is a component of a total hip replacement (THR) in a patient suffering the periprosthetic fracture and wherein the bone engaging portion of the post is adapted to connect with a distal femur below the fracture.

3. The device of claim 2, wherein the clamping portion comprises a cylindrical body with a central cavity sized and shaped to receive the stem, wherein a plurality of threaded openings extending through a wall of the body, the clamping portion further comprising a plurality of bolts threaded into the threaded openings and adapted to be tightened against the stem.

4. The device of claim 2, wherein the clamping portion comprises a first clamshell fixed with the clamp coupling and a second clamshell joined with the first clamshell by one or more bolts, wherein the stem is inserted between the clamshells, and wherein the one or more bolts are tightened to draw the clamshells together to tighten against the stem.

5. The device of claim 1, wherein the clamp coupling and the post coupling are inter-engaging male and female morse taper couplings.

6. The device of claim 4, wherein one or both clamshells include internal features to facilitate the flow of adhesive between the stem and the clamshells.

7. The device of claim 1, wherein the clamping portion further comprises one or more openings adapted to allow an adhesive to be injected into a space adjacent the stem.

8. The device of claim 1, wherein one or both of the clamp and the post include reinforcement receiving openings, wherein the receiving openings are adapted to receive fasteners.

9. The device of claim 1, further comprising one or more adapters, wherein the adapters each comprise first and second couplings at their respective ends.

10. The device of claim 9, wherein the first and second couplings both comprise female morse tapers, or wherein the first and second couplings both comprise male morse tapers, or wherein the first coupling comprises a female morse taper and the second coupling comprises a male mores taper.

11. The device of claim 9, wherein the first coupling defines a first coupling axis, wherein the second coupling defines a second coupling axis, and wherein the first and second coupling axes are at an angle with respect to one another.

12. The device of claim 9, wherein the one or more adapters comprise an elongated central portion having threaded openings at each end, wherein the first and second couplings each comprises a coupling portion and a threaded portion, wherein the threaded portions of the first and second couplings are engaged with the threaded opening, and wherein, when the central portion is rotated relative to the first and second couplings, engagement of the threaded portions causes the couplings to move toward or way from one another.

13. The device of claim 1, further comprising a matrix that is coupled to the stem such that the matrix is between the clamp and the stem.

14. The device of claim 13, wherein the matrix is in the shape of a sleeve that is slid onto the stem.

15. The device 13, wherein the matrix is adapted to be shaped by cutting, compressing, bending, drilling, and burring into a desired configuration.
